(19) United States
(12) Patent Application Publication
(10) Pub. No.: US 2007/0142730 A1
(43) Pub. Date: Jun. 21, 2007

(54) Apparatus for noninvasive blood pressure measurement

(76) Inventors: Franz Laermer, Weil Der Stadt (DE); Gerd Lorenz, Reutlingen (DE); Michael Stumber, Korntal-Muenchingen (DE); Dick Scholten, Stuttgart (DE); Christian Maeurer, Leonberg (DE); Julia Patzelt, Kirchentellinsfurt (DE)

Correspondence Address: KENYON & KENYON LLP, ONE BROADWAY, NEW YORK, NY, 10004, US

(21) Appl. No.: 11/639,052

(22) Filed: Dec. 13, 2006

(30) Foreign Application Priority Data
Dec. 13, 2005 (DE) 10 2005 059 435.2

(51) Int. Cl. A61B 5/02

(52) U.S. Cl. 600/490; 600/500

(57) ABSTRACT

An apparatus for noninvasive blood pressure measurement having a device for measuring blood pressure values and an acceleration sensor, in particular a two- or three-axis acceleration sensor for measuring movements is provided for measuring and monitoring blood pressure. Using the movement data ascertained by the acceleration sensor, movement artifacts are calculated out of the measured blood pressure values with the aid of a signal processing system.

### FIELD OF THE INVENTION

The present invention relates to an apparatus for noninvasive blood pressure measurement having a device for measuring blood pressure values and an acceleration sensor, in particular a two-axis or three-axis acceleration sensor for measuring movements as well as a method for noninvasive blood pressure measurement, a blood pressure value being measured using a device for measuring blood pressure values.

### BACKGROUND INFORMATION

For measuring and monitoring blood pressure, a distinction is made between the noninvasive and the invasive measuring methods. In the invasive technique, a catheter is laid in a large artery of the patient. A transmission medium compressed by the pressure of the blood, such as a physiological saline solution, for example, presses against a pressure transducer, which then generates signals that are proportional to the pressure. The invasive method therefore requires a bodily intervention as well as intact arteries.

For this reason, one falls back on the noninvasive technique, particularly for the occasional monitoring of blood pressure. The methods and devices used for this purpose may be based on initially cutting off the blood flow by external pressure. Subsequently, the pressure is reduced until the systolic blood pressure overcomes the outer pressure. The external pressure is then further reduced until the blood flow is no longer interrupted at any time and the diastolic blood pressure is thus reached or undershot. The external pressure and its variation, for example, may be generated by applying a pressure cuff on an extremity. For ascertaining the blood pressure values, a sound sensor, such as a stethoscope or a microphone for example, is attached near the artery that is closed by pressure. Initially, no sound is yet detected. When the external pressure is reduced, a muted sound, known as the Korotkoff sound, becomes noticeable as the systolic blood pressure is reached. This sound now grows increasingly louder as the external pressure is reduced further until it subsequently becomes fainter again and then disappears entirely. When it disappears, the diastolic blood pressure value is reached. The method is encumbered with measurement inaccuracies, however, which are caused by the difficulty of acoustically perceiving exactly the points of the first appearance and the disappearance of the Korotkoff sound.

In order to avoid these, the so-called oscillometric method is used in blood pressure measuring devices in recent years, as described for example in European Published Patent Application No. 0 642 760, in which the blood pressure is computed directly from the variations in the cuff pressure. In this method, the pulse waves, which are superposed in the measured cuff pressure, are extracted in such a way that the amplitudes of the pulse waves are derived. The pressures, at the points at which the pulse wave amplitude is at a maximum, at which the amplitude is a predefined portion of the maximum value on the side of the higher pressure of the maximum and at which the amplitude is a predefined portion of the maximum pressure on the side of the lower pressure of the maximum, are correspondingly determined as the mean blood pressure, the systolic blood pressure and the diastolic blood pressure.

Generally, this measurement today often occurs with the aid of so-called blood pressure computers. Such a blood pressure measuring device, which takes the form of an electronic armband blood pressure measuring device, is known for example from German Published Patent Application No. 202 19 565.

Starting from the fact that at a higher blood pressure the walls of the arteries are stretched more and the vessel elasticity thus decreases, other noninvasive methods may find a use as well. For with the change of vessel elasticity as a function of the existing blood pressure, the so-called pulse transit time also changes as a function of the blood pressure such that the existing blood pressure of the patient may be inferred by measuring the pulse transit time. To determine the pulse transit time, generally two simultaneous cardiovascular variables have to be measured. For this purpose, for example, pulse waves may be measured continuously in two different locations on an artery branch. The time offset of these pulse waves then corresponds to the pulse transit time. Another possibility is the simultaneous measurement of the heart activity and a pulse wave. This makes it possible to determine the propagation time of the pulse wave from the heart to the pulse wave registering location, which is usually situated peripherally, on a finger for example.

The ECG lends itself for ascertaining the heart activity, although heart sounds or other suitable variables may be measured as well. At the peripheral registration location, the blood flow may be measured with the aid of various methods. In particular, the measurement may occur photometrically, for example with the aid of a pulsoximeter, by impedance measurement, plethysmographically or with the aid of a Doppler method. In choosing the method it is essential that a sufficiently precise ascertainment of the propagation time of the pulse wave is possible.

The measuring results of the known devices, however, are susceptible with respect to movements and vibrations that can occur during the measurement and can interfere with the latter. Often these interferences are interpreted as a pulse and thus result in a falsification of the measuring results. Such interferences occur more frequently in emergency medical service since here ambient noises in auscultatory measurement and movements of the patient are unavoidable and cause so-called movement artifacts. Movement artifacts may significantly falsify a measurement. Such movement artifacts occur in particular if the blood pressure measuring device cannot be held still during the pressure measurement. In addition to the already mentioned emergency medical services, the reasons for this lie in dyskinetic motor disturbances of the patient such as in Parkinson or chorea patients for example. Restlessness of the user or improper operations, which frequently occur in the case of older patients, likewise contribute to the appearance of movement artifacts. This often results in only the systolic blood pressure being measurable and a more extensive measurement on the patient not being possible or the device interpreting the appearing artifacts as Korotkoff sounds and in the end providing false blood pressure values.

To prevent interferences during measurement it is known for example from German Published Patent Application No. 199 02 044 to improve an apparatus for the noninvasive blood pressure measurement by special constructions such as the attachment of a hybrid cable in such a way that faulty measurements can be corrected.

Furthermore it is known from German Published Patent Application No. 20 2004 007 139 to use a three-axis acceleration sensor for correctly positioning the blood pressure measuring device. The position data of the three-axis acceleration sensor are transmitted to a microprocessor, which then analyzes the position data and determines in particular by a comparison whether the blood pressure measuring device is positioned correctly. If this is not the case, then the patient is alerted to the faulty position by a warning sound. The errors that occur due to vibrations or movements, however, cannot be corrected by this means.

### SUMMARY OF THE INVENTION

The object of the present invention is to provide an apparatus for noninvasive blood pressure measurement which makes it possible to improve the measuring accuracy in spite of the movement disturbances.

According to the present invention, this objective is achieved by an apparatus for noninvasive blood pressure measurement. With regard to the method, the objective is achieved in a method for noninvasive blood pressure measurement.

Thus, according to the present invention, initially the blood pressure of a patient is measured noninvasively. For this purpose, for example, an external pressure may be applied, preferably to an extremity of the patient, using a cuff. This pressure applied on the cuff is continuously or periodically measured by a pressure measuring means while the external pressure is being reduced. At the same time, the pressure applied on the cuff is under the influence of pulse waves.

In another example of noninvasive blood pressure measurement, the blood pressure may be measured by measuring the pulse transit time, for example, by using an ECG device and a peripheral sensor. For this purpose, the peripheral sensor may be based on a photometric measurement such as, for example, a pulsoximeter, an impedance measurement, a plethysmographic measurement or a Doppler method. In principle, all other noninvasive methods may also be used for the blood pressure measurement according to the present invention. In all cases, however, an acceleration sensor, in particular a two- or three-axis acceleration sensor for measuring movements continues to be provided. Using an electronic signal processing system, corrected blood pressure values are ascertained from the measured blood pressure values and the measured movement signals of the acceleration sensor. In this manner, the movement during the measurement is taken into account and movement artifacts may be eliminated or at least reduced.

As a simple implementation form, an analog operational amplifier, which is designed as a differential amplifier for example, may be used as an electronic signal processing system. More complex forms include for example digital signal processing systems, microcontrollers and the like.

The acceleration sensor may be implemented as a one-, two- or three-axis acceleration sensor and may be provided on or in the cuff.

The apparatus according to the present invention for noninvasive blood pressure measurement made it possible to ascertain correct blood pressure measurement even during an active or passive movement of the patient and also in emergency use. Movement artifacts, which could be falsely interpreted as a pulse, are filtered out by the signal processing system.

According to the method of the present invention for noninvasive blood pressure measurement, the blood pressure is measured by a device for noninvasive measurement of blood pressure values. With the aid of an acceleration sensor furthermore a possible movement during the blood pressure measurement is measured and movement signals are generated from this. The movement signals and the measured blood pressure values are processed in a signal processing system in such a way that corrected blood pressure values are ascertained, which take a movement during the measurement into account.

Besides the measurement of the blood pressure values using a cuff, another noninvasive blood pressure measuring method may be used as well. In particular, a method may be used, in which the pulse transit time is measured for ascertaining the blood pressure. For this purpose, the blood pressure values may be measured using an ECG measuring device and a peripheral sensor for example. The simultaneous measurement of the peripheral blood flow, particularly by using a photometric method, an impedance measurement, a plethysmographic method or a Doppler method, the pulse transit time may be ascertained, from which the blood pressure values may be derived.

### BRIEF DESCRIPTION OF THE DRAWINGS

FIG. 1 shows a perspective schematic representation of the apparatus according to the present invention for noninvasive blood pressure measurement.

FIG. 2 shows a schematic representation of the method sequence of the propagation time method.

FIG. 3 shows a schematic the sequence of the correction of the blood pressure measurement using a cuff.

FIG. 4 shows a schematic representation of the method sequence of the propagation time method.

### DETAILED DESCRIPTION

The present invention is explained in greater detail in the following with reference to the example of a cuff blood pressure measurement. In this instance, however, the cuff blood pressure measurement is to be regarded merely as an example for a noninvasive blood pressure measurement and is in this respect not to be understood in a restricting manner. For in principle the blood pressure measurement may also occur, as already described, via other noninvasive methods, in particular via one of the already described pulse transit time measurement methods.

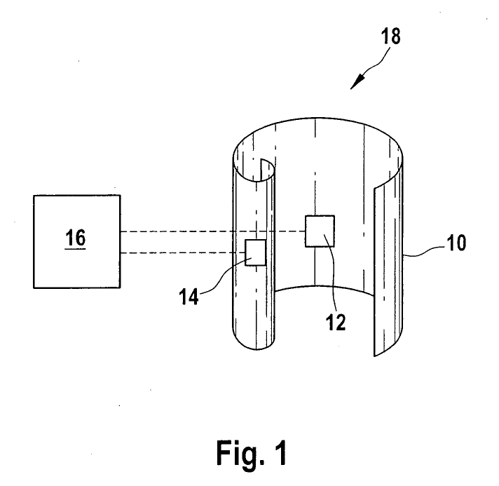

FIG. 1 schematically shows a blood pressure measuring device 18, which has a cuff 10 for generating an external pressure. Cuff 10 is wrapped around an extremity of the patient and is coupled to a pneumatic system such that using it a pressure on a blood vessel of the patient may be generated which is so high as to interrupt the blood flow through the vessel. With the aid of a pressure measuring means 12, for example a pressure sensor, which may be attached to the cuff or be provided separately, the cuff pressure is measured and converted into an electrical signal. Furthermore, an acceleration sensor 14 is provided, which may be provided in or on cuff 10. In this instance, acceleration sensor 14 is designed to be of a one- or multi-axis type. It is capable of being used to measure movements of the patient. Like the signals of pressure measuring device 12, the movement signals are also supplied to a signal processing system 16. In signal processing system 16, the movement artifacts may then be calculated out of the measured pressure signals such that a blood pressure value corrected by the movement may be ascertained. The signal transmission may occur via cables or without cables via IR or radio for example.

Blood pressure measuring device 18 is indeed schematically described with reference to a cuff 10, a pressure measuring means 12 and an operator console (not shown), which in each case are separate from one another. However, blood pressure measuring device 18 may also take the form of a so-called wrist pressure measuring device as described for example in German Published Patent Application No. 202 19 565. In such devices, acceleration sensor 14 may also be attached to the pressure cuff applied on the wrist pressure measuring device or be integrated into it and suitably combined with a signal processing system.

If the blood pressure measurement is performed on the basis of the pulse transit time method, then acceleration sensor 14 may be situated for example on or in the ECG measuring device. Likewise, acceleration sensor 14 may also be situated on or in the peripheral sensor.

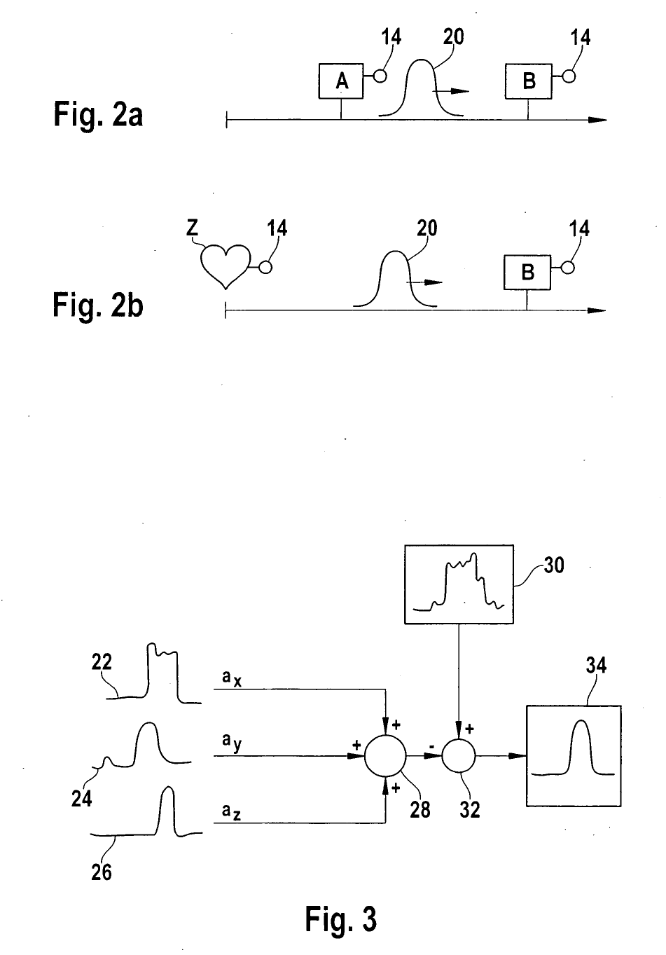

The fundamental method sequence in the propagation time method is represented in FIG. 2. FIG. 2a shows the principle of the propagation time measurement using two peripheral sensors A and B, in which the propagation time of a pulse signal 20 is measured between peripheral sensors A and B and the blood pressure is inferred from this time. In particular pulsoximeters or plethymographs may be used as sensors. Each of the sensors A and B may have an acceleration sensor 14, with the aid of which movements during the measurement may be measured.

FIG. 2b shows a basic representation of the propagation time method using a central sensor Z, for example an ECG or a sonic sensor, which measures the heart sounds directly. In this method, the propagation time between central sensor Z and a peripheral sensor and from that the existing blood pressure value is determined. Acceleration sensor 14 may be provided on central sensor Z as well as on peripheral sensor B.

FIG. 3 schematically shows the sequence of the correction of the blood pressure measurement, which is performed using a cuff and a provided acceleration sensor. In this instance, the acceleration sensor for each of its measuring axes aₓ, a_{y} and a_{z} provides signals 22, 24 and 26, which are additively combined in a signal processing device 28. Signal 30 of peripheral sensor B, that is, in particular the pulse curve or the oscillometric curve is then processed subtractively with the obtained additive signal in device 32 such that a corrected signal 34 results.

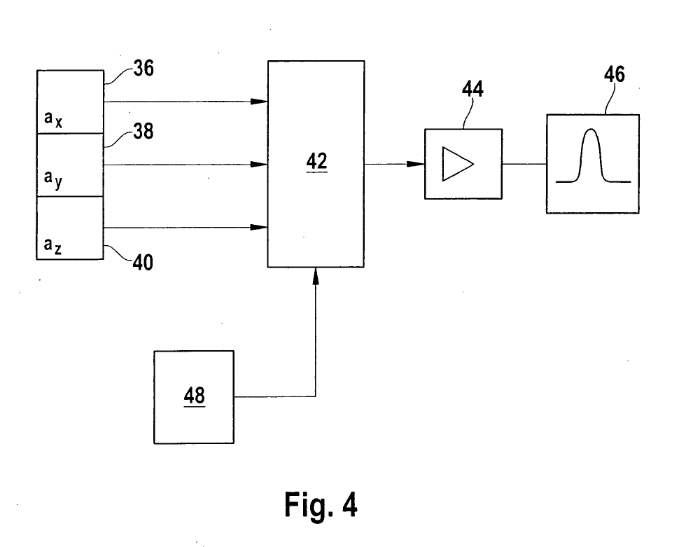

FIG. 4 shows schematically the sequence of the correction of the blood pressure measurement when using the propagation time method together with central sensor Z. The signals of a three-axis acceleration sensor 36, 38, 40 for the three axes aₓ, a_{y} und a_{z} are supplied to a signal processing device 42 in which the signals of the acceleration sensor are summed. Via a filtering of the signal and following the formation of the difference with signal 48 of the central sensor, particularly the ECG, a correction signal 46 cleansed of acceleration artifacts is produced. To improve the signal, an operational amplifier 44 may be provided after signal processing device 42.

What is claimed is:

1. An apparatus for noninvasive blood pressure measurement, comprising:
a device for measuring blood pressure values; and
an acceleration sensor corresponding to a two- or three-axis acceleration sensor for measuring movements, wherein an electronic signal processing system is provided, which is designed in such a way that using it corrected blood pressure values may be ascertained from the measured blood pressure values and the measured motion signals of the acceleration sensor.

2. The apparatus for noninvasive blood pressure measurement as recited in claim 1, wherein the acceleration sensor is used for measuring movements of the device for measuring blood pressure values.

3. The apparatus for noninvasive blood pressure measurement as recited in claim 1, wherein the electronic signal processing system has an operational amplifier corresponding to a differential amplifier.

4. The apparatus for noninvasive blood pressure measurement as recited in claim 1, wherein the electronic signal processing system has a digital signal processing system corresponding to a microcontroller.

5. The apparatus for noninvasive blood pressure measurement as recited in claim 1, wherein the device for measuring the blood pressure values has a cuff and pressure measuring means for measuring a pressure applied on the cuff under the influence of pulse waves and the acceleration sensor is integrated into the cuff.

6. The apparatus for noninvasive blood pressure measurement as recited in claim 1, wherein the device for measuring the blood pressure values has a cuff and pressure measuring means for measuring the pressure applied on the cuff under the influence of pulse waves and the acceleration sensor is situated on the cuff.

7. The apparatus for noninvasive blood pressure measurement as recited in claim 1, wherein the device for measuring the blood pressure values comprises a device for measuring the pulse transit time.

8. The apparatus for noninvasive blood pressure measurement as recited in claim 7, wherein the device for measuring the blood pressure values comprises an ECG device and a peripheral sensor corresponding to a pulsoximeter.

9. The apparatus for noninvasive blood pressure measurement as recited in claim 8, wherein the acceleration sensor is situated on or in the ECG measuring device.

10. The apparatus for noninvasive blood pressure measurement as recited in claim 8, wherein the acceleration sensor is situated on or in the peripheral sensor.

11. A method for noninvasive blood pressure measurement, comprising:
measuring a blood pressure value by a device for measuring blood pressure values, wherein movements are measured using an acceleration sensor and movement signals are generated from this and the movement signals and the measured blood pressure values are processed in a signal processing system in such a way that corrected blood pressure values are ascertained.

12. The method for noninvasive blood pressure measurement as recited in claim 11, wherein the blood pressure values are ascertained using a blood pressure measuring device, an external pressure being applied using a cuff and being reduced and the pressure applied on the cuff being measured using a pressure measuring means under the influence of pulse waves.

13. The method for noninvasive blood pressure measurement as recited in claim 10, wherein the blood pressure values are ascertained with the aid of the method of pulse transit time.

14. The method for noninvasive blood pressure measurement as recited in claim 13, wherein the blood pressure values are ascertained using an ECG measuring device and the measurement of the peripheral blood flow, in particular by using a photometric method, an impedance measurement, a plethysmographic method or a Doppler method.
